(19) United States
(12) Patent Application Publication
(10) Pub. No.: US 2007/0077317 A1
(43) Pub. Date: Apr. 5, 2007

(54) Olive kernel composition containing absorption promoters, antioxidants, and anti-inflammatory agents

(76) Inventor: Theoharis C. Theoharides, Brookline, MA (US)

Correspondence Address: Dr. Melvin Blecher, 4329 Van Ness St., NW, Washington, DC, 20016-5625, US

(21) Appl. No.: 11/519,965

(22) Filed: Sep. 13, 2006

Related U.S. Application Data
(63) Continuation-in-part of application No. 10/811,859, filed on Mar. 30, 2004, now abandoned.

(51) Int. Cl. A61K 36/63

(52) U.S. Cl. 424/769; 554/8

(57) ABSTRACT

The claimed invention is composition comprising an organic extract of de-fleshed, purified, isolated olive kernels that contains one or more components that increase absorption of macromolecules such as proteoglycans across cell membranes, that have antioxidant properties, and that have anti-inflammatory effects in tissues.

This application is a continuation-in-part of U.S. Ser. No. 10/811,859, filed Mar. 30, 2004, which takes its priority from copending PCT/US02/00476, filed Jan. 03, 2002, copending U.S. Ser. No. 09/771,669, filed Jan. 30, 2001 (now U.S. Pat. No. 6,984,667).

### FIELD OF THE INVENTION

The invention is generally related to compositions for the treatment of inflammatory conditions. More specifically, the invention is related to olive kernel organic extracts that contain compositions that promote the absorption of anti-inflammatory agents across biological membranes and that contain antioxidant and anti-inflammatory components.

### BACKGROUND OF THE INVENTION

There have been a number of mostly anecdotal reports that the proteoglycan chondroitin sulfate, as well as glucosamine sulfate, a product of the intestinal breakdown of proteoglycans, may be helpful in relieving the pain of osteoarthritis:—Shute N. Aching for an arthritis cure. US News and World Report, Feb. 10, 1997.—Cowley G. The arthritis cure? Newsweek, Feb. 17, 1997; Foreman J., People, and their pets, tout arthritis remedy. The Boston Globe, Apr. 7, 1997; Tye L. Treatment gains scientific attention. The Boston Globe, Sep. 25, 2000.

A recent meta-analysis showed potential therapeutic benefit of chondroitin sulfate and/or glucosamine in osteoarthritis [McAlindon et al. J Am Med Assn. 283:1469 (2000)], while a double-blind clinical trial with glucosamine showed definite benefits in osteoarthritis with respect to both pain and radiographic joint appearance [Reginster et al., Lancet 337:252 (2001)]. In fact, less than 5% of the chondroitin sulfate in commercially available preparations is absorbed orally, because the size of the molecule and the degree of sulfation impede its absorption from the gastrointestinal tract. Furthermore, such commercial preparations use chondroitin sulfate obtained from cow trachea, with the possible danger of contracting spongiform encephalopathy or “mad cow disease”. In fact, the European Union has banned even cosmetics that contain bovine-derived products.

Theoharides et al. British Journal of Pharmacology 131:1039 (2000) indicated for the first time how proteoglycans such as chondroitin sulfate may work. The paper reported that chondroitin sulfate and, to a lesser degree, glucosamine sulfate, inhibit activation of mast cells that are known to trigger allergy and asthma. This discovery is the basis for Theoharides, U.S. Pat. No. 6,689,748, filed Apr. 8, 1998, and U.S. Pat. No. 6,984,667, filed Jan. 30, 2001.

Mast cells are also now recognized as important causative intermediary in many painflul inflammatory conditions [Galli, N Eng J Med. 328:257 (1993); Theoharides, Int J Tissue Reactions 18:1 (1996)], such as insterstitial cystitis and irritable bowel syndrome [Theoharides, Ann NY Acad, Sci. 840:619 (1998)], as well as in migraines and multiple sclerosis [Theoharides, Persp Biol Med. 26:672 (1983); Theoharides, Life Sci 46:607 (1996)]. In fact, glucosamine was recently considered to be prophylactic for migraines [Russell, Med Hypoth 55:195 (2000)].

Mast cells are increasingly implicated in conditions involving inflamed joints, such as in osteoarthritis and rheumatoid arthritis, through activation of local mast cells by, for example, neuropeptides, such as Substance P. Additional indirect evidence also supports the involvement of mast cells in bone resorption: (a) systemic mastocytosis is invariably associated with osteoporosis; (b) inhibition of mast cell mediator release reversed lytic bone changes; (c) depletion of mast cells inhibited bone resorption in organ culture; (d) human synovial mast cells were shown to secrete in response to allergic and non-immunologic stimuli; (e) human mast cells release the cytokine IL-6 and (f) IL-6 has been definitively linked to bone resorption and osteoporosis.

It was recently shown that chondroitin sulfate's ability to inhibit the activation of mast cells compliments the inhibitory effects on mast cell activation of another class of naturally occurring compounds, the flavonoids [Middleton et al. Pharm Rev 52:1 (2000)]. Certain plant flavones (in citrus fruit pulp, seeds, sea weed) are now recognized as anti-allergic, anti-inflammatory, anti-oxidant and cytoprotective with possible anti-cancer properties. Only some flavonoids that belong to the subclass of flavones, e.g., quercetin, inhibit mast cell activation.

Quercetin inhibits secretion from human activated mast cells [Kimata et al. Allergy 30:501(2000)], and has also been used effectively for the treatment of chronic prostatitis [Shoskes et al., Urology 54:960 (1999)]. However, other flavonoids may have opposite effects. Use of the term “bioflavonoids” or “citrus flavonoids” in certain commercial products, therefore, provides little information, and may include molecules that have detrimental effects; for example, soy contains isoflavones that have estrogen-like activity that worsens inflammatory conditions.

Theoharides U.S. Pat. No. 6,689,748 and divisional Ser. No. 10/439,301 claim the oral use of proteoglycans, without and with flavonoids, for the treatment of mast cell activation-induced diseases. Absorption of these compositions from the gastrointestinal tract and synergism with other treatment modalities were not addressed in these applications.

Theoharides has also described the use of antagonists of the action of Corticotropin Releasing Hormone (also known as Corticotropin Releasing Factor) in inhibiting myocardial mast cell activation in myocardial ischemia (copending U.S. patent application Ser. No. 08/858,136, filed May 18, 1997), in treating stress-induced skin disease (U.S. Pat. No. 6,020,305) and stress-induced migraine headaches (U.S. Pat. No. 5,855,884), the contents of which are incorporated herein by reference. The synergistic effects of the compositions of the present invention that include antagonists of the actions of Corticotropin Releasing Hormone (“CRH”) on mast cells were not recognized at the time of the previous studies. The word “antagonists” in connection with CRH is intended herein to include any molecule that prevents the actions of CRH on target cells, and includes, but is not limited to, anti-CRH neutralizing antibodies or binding proteins, or molecules preventing the release of CRH at local sites (see below for details).

Applicant has also described a method for treating patients with mast cell derived molecules-induced interstitial cystitis with histamine-1 receptor antagonists (U.S. Pat. No. 5,994,357). Treatment of mast cell molecules-induced migraines with histamine-1 receptor antagonists is the subject of Theoharides U.S. Pat. No. 5,855,884. Histamine-3 receptor agonists as pharmaceutical agents in mast cell-involved diseases are described in Theoharides U.S. Pat. No. 5,831,259. The contents of these three patents are incorporated herein by reference. At the time of this invention the synergistic effects of the present compositions with such antagonists had not yet been recognized.

An important need therefore exists for compositions for administration to human patients being treated for mast cell-induced inflammatory diseases by various modalities, that are synergistic in that they have stronger effects than the sum of the effects of the individual components, and also synergistic with conventional clinical treatments of inflammatory conditions. “Synergistic” is also intended to mean: “coordinated or correlated action by two or more structures or drugs” [Stedman's Medical Dictionary, 23rd edition, Williams & Wilkins, Baltimore, 1976]. An important need also exists for formulations that increase the absorption from the gastrointestinal tract, nasal passages and skin surface of the compositions of the invention. Such formulations have been discovered, and are described below.

### SUMMARY OF THE INVENTION

The invention comprises compositions extracted from defleshed and purified olive kernels using hydrocarbon solvents.

In one embodiment, the kernel extract is produced by the steps of: (1) pressing the olives to remove their olive oil content; (2) purifying the intact kernels that remain after the compression step by removing all contaminating olive skins and olive flesh by repeated washing of the kernels with water; (3) drying the purified kernels in a stream of hot air; (4) crushing the dried kernels; (5) extracting the crushed kernels with a hydrocarbon organic solvent and steam; (6) microfiltering or centriguging the extract to remove any remaining particulate matter; (7) heating the extract at 80-100° C. while percolating an inert gas through the fluid to evaporate the solvent; and, (8) storing the kernel extract in the absence of air to avoid oxidation.

In one embodiment, the aforementioned compositions provide improved absorption from the gastrointestinal tract, skin surface, and nasal and pulmonary surfaces of anti-inflammatory molecules such as proteoglycans.

In another embodiment, said kernel extracts contain amounts of simple and complex phenolic compounds that are far greater than those present in olive oil obtained from the flesh of the olive or other plant oils.

In yet another embodiment, olive kernel extracts contain anti-inflammatory compounds far greater than those seen in olive oil or other plant oils.

In still another embodiment, olive kernel extracts contain antioxidant compounds far greater than those found in olive oil or other plant oils.

### DETAILED DESCRIPTION OF THE INVENTION

It has been discovered that a combination of an anti-inflammatory sulfated proteoglycan with one or more of an anti-inflammatory sulfated D-hexoseamine, flavone or isoflavone, CRH antagonist, histamine-1 receptor antagonist, histamine-3 receptor agonist, polyamine, rutin and caffeine has synergistic anti-inflammatory effects when used as a dietary supplement, a topical product or an aerosol for nasal or pulmonary adminstration, without or with a conventional clinical treatment for inflammatory diseases, and that these effects are greatly enhanced by components of a novel olive kernel extract. Within the present context, such inflammatory diseases result from the activation, degranulation and consequent secretion of inflammatory biochemicals from mast cells, and the resultant inflammatory diseases include the group consisting of: allergic inflammation, arthritis (to include osteoarthritis and rheumatoid arthritis), fibromyalgia, inflammatory bowel disease, interstitial cystitis, irritable bowel syndrome, migraines, atherosclerosis, coronary inflammation, ischemia, chronic prostatitis, eczema, multiple sclerosis, psoriasis, sun burn, periodontal disease of the gums, superficial vasodilator flush syndromes, hormonally-dependent cancers, endometriosis and medical devices. The olive kernel composition alone may be used to improve the transmembrane transport of difficultly-absorbable drugs in the intestine, skin and pulmonary alveoli.

In a highly preferred embodiment, the sulfated proteoglycan is non-bovine chondroitin sulfate, preferably from shark cartilage, which blocks mast cell activation, degranulation and consequent secretion of inflammatory biochemicals from the mast cells. Other natural sulfated proteoglycans suitable for practicing this invention include keratan sulfate, dermatan sulfate and hyaluronic acid sodium salt (sodium hyaluronate). The preferred biological source of the chondroitin sulfate is shark cartilage which is more-highly sulfated than the common commercial chondroitin sulfate isolated from cow trachea; the shark cartilage source also avoids the potential dangers associated with bovine sources.

Certain flavones inhibit secretion of inflammatory molecules from mast cells by affecting moesin, a unique 78 kDa mast cell protein [Theoharides et al. J Pharm Exp Therap 294:810 (2000)]. Flavones suitable in carrying out the invention include: quercetin, the quercetin glycoside rutin, myricetin, genistein, kaempferol, the isoflavone phenoxodiol, and the kaempferol glycoside astrazaline. The flavonide luteolin and its derived glycosides are are also known to be anti-inflammatory compounds (Odontuya, G., et al. Phytother. 19: 782 (2005)) and, as will be demonstrated in the Examples, the present inventive olive kernel composition contains higher levels of luteolin than the olive from which it was derived.

The inventive olive kernel extract composition is produced from, preferably, Greek olives, particularly those produced on the island of Crete. This kernel extract is especially prepared by applicant's inventive process consisting essentially of: (1) pressing the olive flesh to remove its oil content; (2) purifying the kernels that remain after the compression step by removing all contaminating skins and pulp(flesh) by repeated washing of the kernels with water (this is a critical step); (3) drying the washed kernels in a stream of hot air; (4) crushing the purified, dried kernels; (5) extracting the crushed kernels with an organic solvent (such as the hydrocarbon hexane and the like) and steam; (6) microfiltering or centtrifuging the organic extract (1-2 micron pore size for microfilters) to remove any particulate matter; (7) heating the organic extract at 80-100° C. while percolating an inert gas such as helium (to avoid oxidation) through the fluid to evaporate the solvent (final concentration <1 g %), which process reduces the water content to <1 g % and the acidity as oleic acid of <1 g %; and, (8) storing the kernel extract in the absence of air to avoid oxidation.

The inventive olive kernel extract (herinafter OKE) described above not only has endogenous anti-oxidant and anti-inflammatory properties, but surprisingly has the unique property of increasing absorption of the other components of the anti-inflammatory compositions through the intestinal mucosa or skin or pulmonary alveoli (see Example, below).

Olives are known to contain several simple and polymeric phenolic compounds, not found in other vegetables, that are powerful antioxidants useful in human metabolism. These include simple phenolic compounds such as hydroxytyrosol, clenoic acid hydroxytyrosol and oleuropein aglycone, and the polymer polyphenol.

It is known that the total phenolic content best correlates with antioxidant activity (Del Carlo, M. et al. J Agr Food Chem 52:4072 (2004)). We found that the average total phenolic content (determined by the colorimetric method of Singleton, V L et al., Am J Enol Vitic 16:144 (2005)) of the inventive OKE was 1.650 mg/kg, while that of virgin olive oil obtained from the flesh of the olive was 590 mg/kg. Thus, the kernel of the olive is a far better source of antioxidant phenols than is the flesh of the olive.

The polyphenols found in such OKE also have anti-inflammatory effects in, for example, arthritis (Martinez-Dominguez et al., Inflamm. Res. 50:102 (2001)). As will be detailed ibelow, the inventive OKE exhibited anti-inflammatory effects in a cytokine system to a much larger extent than virgin olive oil. Corn oil was without effect.

In addition to its usefulness in increasing the absorption of the inventive macromolecular compositions across the intestinal wall and the skin, we have found that the inventive OKE is useful in aiding the absorption of other difficultly-absorbable drugs, e.g., the HDL-increasing drug torcetrapib (escribed by DeNinno et al. U.S. Pat. No. 6,586,448).

Supplementation of the compositions described above with the methylation reagent S-adenosylmethionine (“SAM”) adds antioxidant, anti-inflammatory and cytoprotective properties, particularly in inflammatory joint diseases. Addition of SAM also accelerates metabolism of homocysteine, which amino acid has been implicated in coronary disease, to cysteine, which is harmless. Folic acid may be added to certain of the present formulations for similar reasons.

Another supplement to the basic compositions of the invention is a histamine-1 receptor antagonist, such as hydroxyzine, merelastine, azelastine, azatadine and cyproheptadine. Other histamine-1 receptor antagonists are described in Table 25-1 in Goodman and Gilman's The Pharmaceutical Basis of Therapeutics, 9^{th} ed., New York, 1996. Histamine-3 receptor agonists are described in the Theoharides patents listed above.

Inhibitors of mast cell activation and secretion of inflammatory biochemicals may be used in the treatment of inflammatory processes such as superficial vasodilator syndrome, such as occurs in menopausal-associated flush, carcinoid flush, MSG-associated flush, and niacin-associated flush.

Hormone-dependent cancers, including the estrogen/progestin linked ovarian, uterine, breast, and endometrial cancers, and the androgen-linked testicular cancers, are associated with tissue inflammation. These inflammations can be treated with chondroitin sulfate, quercetin, genestein, phenoxodiol isoflavone, olive kernel oil/extract, and, optionally, chemotherapeutic agents such as tomoxifen or raloxifen.

The inventive compositions may also be used as coatings on implanted medical devices, which devices may lead to or be associated with inflammation of surrounding tissues, in order to provide protection against such inflammations. Not only can the coating of such medical devices inhibit or protect against inflammation caused by the device itself, but the coated devices can also be used to deliver the inventive compositions to innately inflamed tissues due to other causes. Such medical devices include artificial skins (scaffolding such as naturally occurring polymers, e.g., collagen; man-made polymers, e.g., PTFE, Dacron, PET or polyethylene; self-degrading man-made polymers, e.g., PLA or PGA; biopolymer matrices from animal tissues including fetal and neonatal tissues to be used as tissue engineering scaffolds (cf. Bell et al., U.S. patent application Pub. No. 20020146393)), artificial joints, band-aids, stents for blood vessels, artificial blood vessels, pacemakers, stents for abdominal support in hernia repair, tissue transplants, prostheses, breast implants, etc. Particularly useful in this regard are compositions containing heavily sulfated, non-bovine proteoglycans (e.g., chondroitin sulfate) and flavonoids (e.g., quercetin, myricetin, gentistein) with the inventive OKE.

Sources of CRH antagonists include, in addition to the Theoharides patents listed in the Background section above: Neurocrine Biochem. Inc.'s D-Phe 12 Nle Ala32,21,38hCRH(12-41)NH2, cat no. 1P-36-41; Pfizer non-peptide CP-154,526-1; Sigma Chem., St. Louis anti-CRH polyclonal antiserum; and Pfizer, NY patents and applications: U.S. Pat. No. 6,211,195, U.S. Pat. No. 5,795,905, PCT/IB95/00573, PCT/IB95/00439, U.S. Ser. No. 08/448,539, U.S. Ser. No. 08/481,413, U.S. Ser. No. 09/735,841, and in Owens et al. Pharm. Rev. 43:425 (1991).

The preferred concentration range of the proteoglycan, hexosamine sulfate and flavone components of the oral formulations are 10-3,000 mg per tablet or capsule. The preferred concentration range for SAM is 3-1,000 mg per capsule or tablet. Generally, the optimum amounts of the inventive OKE are at least three times those of the other active ingredients, preferably 300-1200 mg. The number of capsules or tablets to be taken per day is determined by the nature and severity of the medical condition, and is readily determinable by the patient's health provider. Other representative formulations are described in the examples below.

The compositions of the invention may be formulated in any standard means of introducing pharmaceuticals into a patient, e.g., by means of tablets or capsules. The compositions of the invention include ointments and creams for skin conditions, mouth washes and toothpaste for periodontal diseases, and solutions for nasal aerosols. Standard excipients and carriers for the active ingredients of the inventive compositions are described in Remington's Pharmaceutical Sciences, Mack Publishing Co., Easton, Pa.

### EXAMPLES

### Example 1

Table 1 compares chondroitin sulfate-containing commercial products to the present compositions.

TABLE 1
Comparison of Chondroitin Sulfate-Containing
Products to Present Invention

| | Most Available | |
|---|---|---|
| Product | Compositions | Present Invention |
| Main ingredient | Mixture of | Non-bovine chondroitin |
| | chondroitins | sulfate, preferably the |
| | | C type |
| Source | Cow trachea | Non-bovine, e.g., shark |
| | | cartilage |
| Amount per capsule | 100-300 | 10-3000 mg |
| or tablet | | |
| Degree of sulfation | Low, if any | High |
| Absorption from | <5% | >15% (higher with OKE) |
| g.i. tract | | |
| Target | Unknown | Mast cells, inflammatory |
| | | cells |
| Other | Vitamins, fish oils | Flavones, OKE, SAM, |
| ingredients | (some preparations) | histamine-1 receptor |
| | | antagonists, histamine-3 |
| | | receptor agonists, CRH |
| | | antagonists, polyamines, |
| | | caffeine, folic acid |
| Advantages | None known | Anti-allergic, anti- |
| | | inflammatory, anti- |
| | | oxidant, cytoprotective |
| Adverse effects | Risk of mad cow | None known |
| | disease, spongiform | |
| | encephalopathy, | |
| | stomach upset, allergy | |
| | to fish products | |
| Relevant | Osteoarthritis | Allergic inflammation |
| conditions | | angina, asthma coronary |
| | | artery disease, arthritis |
| | | (osteoarthritis or |
| | | rheumatoid arthritis), |
| | | chronic prostatitis, |
| | | eczema, fibromyalgia, |
| | | interstitial cystitis, |
| | | irritable bowel syndrome, |
| | | inflammatory bowel |
| | | disease, migraines, |
| | | multiple sclerosis, |
| | | psoriasis, periodontal |
| | | disease, flush syndrome, |
| | | cancer (including |
| | | hormonally-dependent |
| | | forms). |
| Scientific | None found | Theoharides et al. Br J |
| publications | | Pharm 131: 1039 (2000) |
| | | Middleton et al. Pharm |
| | | Rev 52: 673 (2000) |

In all examples, chondroitin sulfate is to assumed to be of a non-bovine variety.

### Example 2

Composition For Protecting Against Inflammatory Diseases
Two capsules to be taken orally 2-3 times daily, at
least one hour before meals

| Ingredients, per capsule, | mg: |
|---|---|
| Chondroitin sulfate | 150-300 |
| D-Glucosamine sulfate | 150-300 |
| Quercetin | 150-300 |
| OKE | 350-1200 |

### Example 3

Composition For Protecting Against Arthritis

| Ingredients per capsule, | mg: |
|---|---|
| D-Glucosamine sulfate | 150-300 |
| Chondroitin sulfate | 150-300 |
| Sodium hyaluronate | 100-200 |
| Quercetin | 150-300 |
| OKE | 350-1200 |

### Example 4

Topical Composition For Protecting Against Arthritis
Skin ointment or cream. Apply three times per day to affected areas.

| Ingredients | % by weight |
|---|---|
| D-glucosamine sulfate | 5 |
| Condroitin sulfate | 5 |
| Sodium hyaluronate | 0.5 |
| Bitter willow bark extract | 5 |
| Quercetin | 3 |
| Aloe vera | 10 |
| OKE | 5 |

### Example 5

Composition For Protecting Against Cardiovascular Disease

| | mg/capsule: |
|---|---|
| Chondroitin sulfate | 50 |
| Kaempferol | 100 |
| S-adenosylmethionine | 50 |
| Niacin | 0.01 |
| OKE | 350-1200 |
| Bitter willow bark extract | 5% by weight |
| Polyunsaturated fatty acids(DHA, DPA) | 100-600 |

### Example 6

Oral Composition For Protecting Against Inflammatory
Processes in Relapsing Multiple Sclerosis

| Ingredients, | mg/day |
|---|---|
| Chondroitin sulfate | 50-300 |
| Quercetin or myricetin | 50-300 |
| Hydroxyzine | 50-300 |
| OKE | 350-1200 |
| Optionally, interferon-beta | 8 million IU Betaferon (Schering), |
| | s.c., on alternate days or 30 μg |
| | (Avonex, Biogen) i.m. once weekly |
| Optionally, a CRH receptor | 5 |
| antagonist | |

### Example 7

Composition For Protecting Against Cystitis And Prostatitis

| Ingredients, | mg/capsule or tablet: |
|---|---|
| D-glucosamine sulfate | 50 |
| Chondroitin sulfate | 100-300 |
| Sodium hyaluronate | 200 |
| Quercetin | 100-400 |
| OKE | 350-1200 |

### Example 8

Composition For Protecting Against “Flush”

| Ingredients, | per capsule: | |
|---|---|---|
| Chondroitin sulfate | 50 | mg |
| Quercetin | 150-350 | mg |
| OKE | 100-750 | mg |
| Bitter willow bark extract | 5% by weight | |
| Optionally, cyproheptadine or azatadine | 4 | mg |

### Example 9

Cream Composition For Protecting Against Skin Allergy

| Ingredients: | % by weight |
|---|---|
| Aloe vera | 5 |
| Non-bovine chondroitin sulfate | 5 |
| Myricetin | 5 |
| Alpha-tocopherol | 5 |
| OKE | 5 |
| Aloe vera | 10 |
| Optionally, azelastine or hydroxyzine | 5 |

### Example 10

Composition For Protecting Against
Hormonally-Dependent Cancers

| Ingredients, | mg/day |
|---|---|
| Chondroitin sulfate | 50-300 |
| Quercetin | 25-250 |
| Genestein | 50-300 |
| Phenoxodiol isoflavone | 500-1000 |
| OKE | 350-1200 |
| Optionally, tomoxifen or raloxifen | About 10 |

### Example 11

### Effect of Olive Kernel Extract on Absorption of a Proteoglycan Sulfate

### In Vivo

Chondroitin sulfate was tritiated by New England Nuclear Corp. to a specific activity of 4.3 mCi/ml.

Unlabeled chondroitin sulfate was dissolved in OKE at a ratio of about 55 w/v chondroitin sulfate powder to about 450 w/v of olive kernel extract (2.9% acidity as oleic acid, 1.03% water, 0.08% hexane). To this solution was added 20.2 microcuries of the labeled chondroitin sulfate. AAA gelatin capsules were filled with the resulting solution using an aluminum template molding device.

The laboratory animals (250 g male Sprague-Dawley rats) were kept overnight without food but with free access to water. One capsule containg the above-described chondroitin sulfate-OKE solution was given to each rat per os. Control animals were given the equivalent amount of chondroitin, but without olive kernel extract. The animals were then given free access to food. Serum radioactivity was measured 8 hours thereafter in a beta scintillation counter.

The results showed that, in control animals, about 3.9%±0.4% (n=3) of the dose of labeled chondroitin sulfate reached the circulation. In sharp contrast, in animals given the OKE along with the labeled chondroitin sulfate, about 14.3%±0.7% (n=4) of the dose was absorbed into the general circulation.

These results demonstrate that OKE increased by almost 400% the absorption of a proteoglycan from the intestine into the general circulation.

Parallel experiments with codfish oil, corn oil and olive oil (from the flesh of the olive) could not be performed as chondroitin sulfate solubility in these oils was insufficient to meet the requirements of the experiment.

### Example 12

### Anti-Inflammatory Effects of Inventive Olive Kernel Extract

We tested the ability of OKE, virgin olive oil and corn oil for their ability to inhibit the release from cultured human mast cells of the pro-inflammatory cytokine, Interleukin-6, in response to stimulation by Interleukin-1 (method of Kandere-Grzybowska, K. et al., J Immun 171:4830 (2003)).

OKE inhibited Interleukin-6 release from mast cells by at least 40%. Corn oil and virgin olive oil had no effect.

If quercetin or glucosamine sulfate (each 1 μM) were also present along with the OKE, the inhibition of Interleukin-6 release was almost complete (95%).

These results are seen in Table 2 in which human leukemic mast cells were preincubated with the oil or OKE and/or compound shown for 10 min at 37° C. before stimulation by IL-1 (50 ng/ml) for 12 hrs. IL-6 was measured by ELISA.

TABLE 2

| Conditions | IL-6 produced (pg/10⁶ cells) |
|---|---|
| Basal | 25 |
| IL-1 alone | 632 |
| Corn oil aline | 680 |
| Olive oil alone | 510 |
| OKE | 393 |
| Quercetin, 1 μM (“Q”) | 280 |
| Chondroitin sulfate, 1 μM (“CS”) | 423 |
| OKE + Q | 125 |
| OKE + CS | 340 |
| OKE + Q + CS + glucosamine | 89 |

### Example 13

### Luteolin Composition of Olive Components

Luteolin Purity Assay

Samples were assayed for luteolin content and purity by high performance liquid chromatography (HPLC) with detection by both ultraviolet (UV) absorbance and mass spectrometry (MS). A known amount of sample (10 mg) was dissolved in 10 mL of pure dimethylsulfoxide and mixed. Ten uL of this solution was then diluted to 1 mL in HPLC mobile phase (0.5% formic acid and 10% acetonitrile in water). 20 uL (200 ng) was then injected into the HPLC system, which consisted of a Thermofinnigan Surveyor HPLC system with UV diode array, Phenomenex Synergi-RP 2×150 mm column, and Deca XP Plus Ion Trap mass detector with Electrospray Ion Source operated in positive ion mode. Mobile phase was run at 0.4 mL/minute with a gradient from 10% acetonitrile to 100% acetonitrile (balance 0.5% formic acid in water) over 20 minutes. The identity of unknown HPLC peaks as luteolin was determined by UV absorbance spectra (Lambda max at 255 and 350 nm), mass spectrum (single M/Z+H peak at 287.2 mass units), and retention time (7.88 minutes), which were identical to pure standard obtained from Sigma-Aldrich (#L9283). Purity (%) was calculated by comparison of the peak heights at 350 nm UV absorbance of unknowns to the standard from triplicate determinations (given in Table 3).

TABLE 3

| | Estimated | 200 ng on column at 350 nm UV absorbance | | |
|---|---|---|---|---|
| | Purity | Peak | Peak | Peak |
| Olive fraction | ww % | height 1 | height 2 | height 3 |
| Luteolin stnd. | 100% | 172,000 | 159,000 | 169,000 |
| Olive oil | 1.6% | 3,800 | 2,270 | 1,980 |
| OKE | 5.3 | 12,600 | 7,260 | 6,590 |
| Crude kernels | 2.6 | 6,200 | 3,660 | 3,340 |

It is clear from these data that olive kernels have a higher content of luteolin than the olive flesh from which the kernels stemmed.

I claim:

1. An olive kernel composition comprising kernel components extracted from de-fleshed, purified olive kernels by a hydrocarbon organic solvent.

2. The composition of claim 1 produced by the steps of: (1) pressing the olives to remove the olive oil content from the flesh; (2) purifying the intact kernels that remain after the compression step by removing all contaminating skins and pulp by repeated washing of the kernels with water; (3) drying the washed kernels in a stream of hot air; (4) crushing the isolated, dried kernels; (5) extracting the crushed kernels with an organic solvent and steam; (6) microfiltering or centrifuging the extract to remove any particulate matter; (7) heating the extract at 80-100° C. while percolating an inert gas through the fluid to evaporate the solvent; and, (8) storing the kernel extract in the absence of air.

3. The composition of claim 2, wherein said hydrocarbon solvent is selected from the group consisting of hexane, heptane or octane.

4. The composition of claim 2, wherein said kernel extract passes through a 1-2 micron pore size filter.

5. The composition of claim 2, wherein said organic solvent and water are removed at a temperature of 85-100° C.

6. The composition of claim 2, wherein the final organic extract composition contains <1% water, <1% acidity as oleic acid, and <1% of solvent.

7. The composition of claim 1, wherein said composition contains simple and complex anti-oxidant phenols.

8. The composition of claim 8, wherein the total amount of said anti-oxidant phenols is greater than that of olive oil orolive pumice oil.

9. The composition of claim 8, wherein said anti-oxidant phenol is luteolin, and said luteolin is present in olive kernels in an amount at least twice that of olive oil.

10. The composition of claim 1, wherein said composition has an anti-inflammatory effectiveness greater than that of olive oil or olive pomice oil.

11. A method of increasing the absorption across tissue semipermeable membranes of a subject of a difficultly-absorbable anti-inflammatory composition, comprising the step of admixing said anti-inflammatory composition with the composition of claim 1 in clinically effective proportions, prior to administration of said anti-inflammatory composition to a subject.

12. The method of claim 11, wherein said anti-inflammatory composition contains a proteoglycan.

13. The method of claaim 12, wherein said proteoglycan is non-bovine, highly sulfated chondroitin sulfate.

14. The method of claim 11, wherein said anti-inflammatory composition contains a flavonoid compound.

15. The method of claim 14, wherein said flavonoid compound is luteolin.

16. The method of claim 11 wherein said semipermeable is selected from the group consisting of the intestinal mucosa, the skin or pulmonary alveoli.
